(19) United States
(12) Patent Application Publication
(10) Pub. No.: US 2022/0117505 A1
(43) Pub. Date: Apr. 21, 2022

(54) Method for Health Detection Supportive Devices and System

(71) Applicant: MEGATHERAPY LTD, Kiryat Ono (IL)

(72) Inventors: Dror HAREL, Kiryat Ono (IL); Shay SHARGAL, Petach TIkva (IL)

(21) Appl. No.: 17/500,969

(22) Filed: Oct. 14, 2021

(30) Foreign Application Priority Data
Oct. 16, 2020 (IL) 278099

(51) Int. Cl. A61B 5/024; A61B 5/01; A61B 5/00; A61B 5/25

(52) U.S. Cl. CPC A61B 5/02427; A61B 5/01; A61B 5/6824; A61B 5/25; A61B 5/742; A61B 5/746; A61B 5/0002

(57) ABSTRACT

A system is provided for managing medical data pertaining to a plurality of users. The system has a sensor unit suitable for obtaining sensed data each time from a given single user and the sensed data is of at least one parameter representative of a medical condition of a given user. The system further has a processing unit for comparing the at least one measured parameter to base line data relating to the same given user.

### FIELD OF THE INVENTION

Embodiments of the invention relate to systems and processes for managing and measurement/detection of data relating to group of medical/biological conditions of a plurality of subjects (such as patients).

### BACKGROUND OF THE INVENTION

Traditionally, many modern programmable medical devices, for example, ECG systems, include internal memory for generating and storing data representing actual device operation over a period of time. The stored data may be reviewed from the medical device on a periodic basis by medical personnel, so that the subject's condition and treatment regimen can be closely monitored, and the medical device may be reprogrammed as needed. However, to retrieve data from certain prior medical devices, such as ECG system, the subject would have been required to make regular visits to the same medical treatment facility. As well, the memory of any market medical device is limited.

To overcome this drawback, raw data has been transferred from medical devices, manually or as copied files, to other data storage and/or processing device. Such personal medical file is common in many healthcare system and files can be approached by the health care provider branches, private doctors, hospitals or other confirmed bodies.

While some systems have been developed for retrieving subject information from a subject's medical device, there is a need in the industry for a device/system that will enable a fast and easy approach to critical medical/biological parameters, which will possibly not be depended on the internet, computers and the slow approach to information, if valid at all for the specific patient.

Such a system may enable a fast real time test of the recorded standard parameters, which can be compared to the base line in order to get fast diagnosis or alerts for further and deeper medical check.

In a case base line data is not available; the system still shall be able to provide the real time tested parameters without showing the recorder base line parameters.

As well, the system shall enable transferring the base line and real time parameters via a mobile phone application or the internet, for the evaluation of professional medical personnel.

Advantage of such a system is clear for any kind of remote or local health care, emergency health care, Nursing homes, personal health care, etc.

The system may provide the best results available for a fast (e.g. maximum about 60 seconds) test, as close or equal to medical gold standards. The quantity of parameters and given results, as well as the quality of analyzing algorithms, may be of a much higher level than market wearable or home using test devices can offer.

### SUMMARY OF THE INVENTION

Embodiments of the invention may relate to medical data measurement and possibly comparing such data measurement to a given base line data, related to the same person.

In at least certain embodiments, the present invention may be defined as relating to systems and processes for managing data relating to group of medical/biological conditions of a plurality of subjects (such as patients).

In certain cases, such systems and processes may be based on comparison in between base line or previous detected values of parameters to real time detected parameters.

Embodiments of such systems and processes may provide various functions for several types of users, including patients or subject-users, healthcare provider-users and payor entity-users and combinations thereof, which allow for improved treatment and medical data management of individual subjects and groups of subjects and which allow collection and analysis of aggregate data from many subject sources, for improving overall healthcare practices of providers and subjects (e.g., patients).

In at least certain embodiments, data measurement shall be done via several sensors which may then processed for analyzing the received data. Such data measurements may relate to a list of medical or biological parameters that can depict a medical conditions of a person.

Base line information shall include a similar list of parameters to those relating to data measurements, and may include identification (ID) information that may enables access to fields for parameters or special personal notifications, due to the person's known medical facts.

In certain cases, interior aspects of a wrist of a person, possibly a right hand wrist, shall be used for an easy and fast measurement of parameters during real time event, while a middle or index finger, e.g. of a left hand, may be used for closing a measurement circuit, such as an ECG measurement circuit.

It is noted that reference herein made to interior aspect of wrist, refers to an interior/anterior aspect/region of the wrist, possibly a region bridging the hand to the forearm.

Embodiments of such systems may require the recording of the base line parameters. Base line information shall be recorded on a token device as an NFC type coin or on a Mobile Phone or other devices which include compatible NFC writing/reading facility.

According to at least certain embodiments of the present invention, a medical data measurement, analyzing and comparing system shall be configured with a group of software algorithms loaded and running on the internal MCU (CPU) of the system.

In at least certain embodiments, functions and applications of the subject system, shall be embodied as including at least some of the following: a sensor unit, possibly a tabletop type agronomic sensor unit, providing e.g. three types of sensor analyzed data and information. In an embodiment, the sensor unit may be connected via a cable to an external device which can also charge the sensors unit battery, or, to a mobile device by BT (Bluetooth) transmission of the information.

Recording of parameters on a token or mobile may be accomplished by a computer, sending information which may have been collected from gold standard medical equipment or data due from sensors unit of the system.

A Read/Write NFC RFID unit, with matching display shall be manufactured for the subject system. Deploying of a full system activity, may in some cases include the Read/Write NFC and a display device for presenting the real time sensors results, analyzed and calculated, displayed possibly in several display sections (e.g. two pages), in comparison to the recorded base line parameters as recorded on a token or mobile phone.

An additional display section (e.g. a third page of subject system), shall display real time ECG plot and potential warnings analyzed by an algorithm, based on data provided by the ECG plot and parameters.

Users may communicate with a medical facility over the Internet, for example, using a conventional personal computer (PC or other suitable network device) having conventional browser software and/or other software for interacting with the system. In addition, real time and base line information may possibly be sent via a mobile phone which is loaded with a computer program or software application enabling functionally of various aspects of the system of the present invention.

Embodiments of such systems and processes may provide various functions for subject-users, healthcare provider-users and payor entity-users and combinations thereof, for improved treatment and medical data analyzing and management for individual subjects and/or groups of subjects.

According to at least certain embodiments, three types of sensors shall be used for real time measurements.

In certain cases, temperature of a patient may not be a base line recorded parameter, but nevertheless may be measured by an accurate built-in sensor possibly with a maximal tolerance of about 0.1 Celsius degrees.

A Photoplethysmogram (PPG) sensor complex, based on reflection of e.g. three types of LED lights; Red, Green and IR, shall be used for part of the parameters measurements. In certain embodiments, three electrodes, Left, Right and RLD (Right Leg Drive, as a reference) shall be used for the ECG plot and derived measurements. Part of the parameters may be calculated by using algorithms, based on data received from both ECG and PPG types of sensors.

In certain embodiments, unique secured algorithms may be comprised as a part of the embodiment of the system. Such algorithms may be arranged for providing numeric parameters from the ECG plot as well the warnings of diseases due to an analyzed ECG. Algorithms may be arranged to meet a better than 2% accuracy as in common gold standard devices.

### BRIEF DESCRIPTION OF THE DRAWINGS

The novel features of the subject matter disclosed herein are set forth with particularity in the appended claims. A better understanding of the features and advantages of the subject matter disclosed herein will be obtained by reference to the following detailed description that sets forth illustrative embodiments, in which the principles of the subject matter disclosed herein are utilized, and the accompanying drawings of which:

FIG. 1 illustrates sensors and calculating components according to an exemplary embodiment of the present invention;

FIG. 2A illustrates a view of using the sensors component/unit according to an embodiment of the present invention, and FIG. 2B illustrates a perspective top view of an embodiment of a sensors component/unit;

FIGS. 3A and 3B illustrates cross sectional and perspective views, respectively, of the sensors component/unit according to an exemplary embodiment of the present invention;

FIGS. 4A to 4C illustrate views of ECG Electrodes according to an embodiment of the present invention;

FIG. 5 illustrates page 1 to 3 of an exemplary system parameters, plot display and warnings due to results analyze, as shown on system display, according to an embodiment of the present invention;

FIG. 6 illustrates the NFC Write/Read only component according to an embodiment of the present invention;

FIG. 7 illustrates the complete system description according to an embodiment of the present invention;

FIG. 8 is a flow chart of the complete system use according to an embodiment of the present invention;

FIG. 9 is a flow chart of central algorithms MCU according to an exemplary embodiment of the present invention;

FIG. 10 illustrates the NFC device read/write and display exemplary component according to an embodiment of the present invention;

FIG. 11 Illustrates the NFC data presented on a writing computer according to an exemplary embodiment of the present invention; and

FIG. 12 illustrates the 5 mobile phone pages according to an exemplary embodiment of the present invention.

### DETAILED DESCRIPTION OF THE INVENTION

The illustrative exemplary embodiments provide a method and system for fast, user friendly, noninvasive and low cost, collection of important medical parameters. Parameters shall be compared to base line recorded parameter of the patient. Parameters can be analyzed without base line if such information is not available or be sent for external evaluation by any known art. As well, the recorded parameters can be used as “stand alone” information without using the offered embodiment of the parameters collection by the full system sensors.

Deploying of the full system capability in at least certain embodiments may be based on the use of two different devices. Such devices are described hereby as sensor unit 1 and processing unit 2.

Sensor unit 1 may be used in conjunction with a processing unit 2 of a system embodiment. Sensor unit 1 in certain embodiments may be used as a standalone device which can send the information to processing unit 2 for remote recording and/or displaying of data. In some non-binding examples, processing unit 2 may be embodied as a mobile phone, a remote computer (or the like).

In at least certain embodiments, sensor unit 1 may include three types of sensor complexes. In one example, such sensor complexes may include analog front-end (AFE) chips such as Texas Instruments AFE4900, Maxim Integrated MAX30003 and MAX86171, and sensors such as Osram SFH7072 and Texas Instruments TMP117 (and the like).

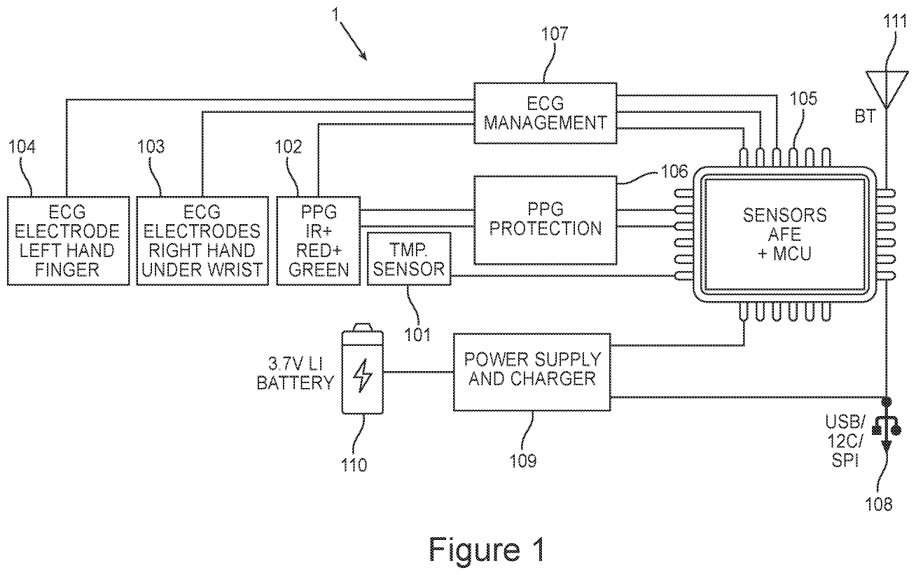

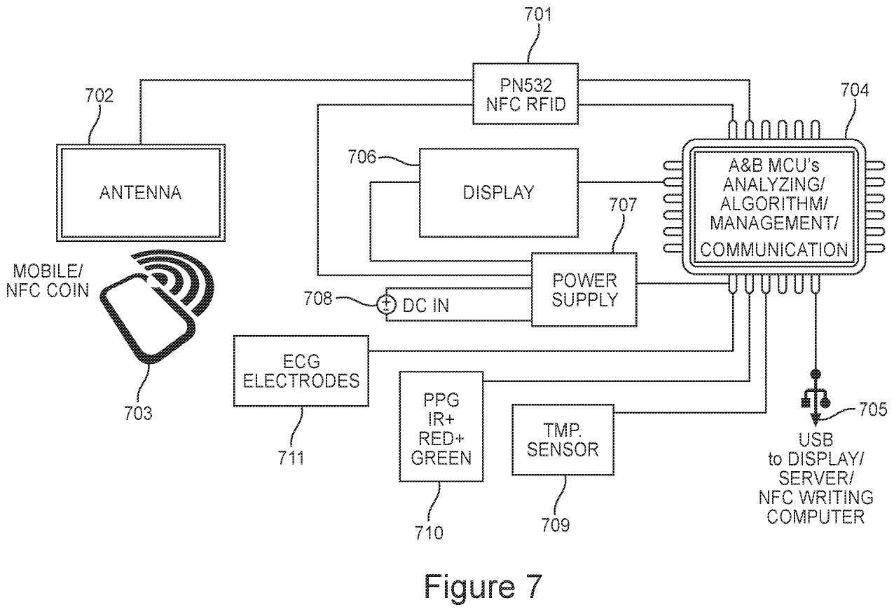

Additional components that may be comprised in sensor unit 1 may include: a high grade Analog Front End (AFE) device, a processor (such as an ARM Cortex-M7 MCU with Data Cache and memory), a local power supply with rechargeable battery (etc.). Embodiments of sensor unit 1 can be seen in FIGS. 1 to 4 and as part of FIGS. 7 to 9.

Processing unit 2 may also comprise a write and read device for NFC RFID devices and a display for recorded data/information. As part of a complete system embodiment, processing unit 2 may be arranged to receive data/information from sensor unit 1 and may be configured to display such sensed results aside data/information possibly obtainable from the NFC read (in embodiments including an NFC device).

In certain embodiments, processing unit 2 may be used for recording data/information (e.g. sensed data/information obtained by sensor unit 1). For example, processing unit may record such data/information to an NFC coin or other known NFC selected item or to a mobile device which offers the NFC service.

In addition, processing unit 2 may be used as a standalone reader device to be installed in any type of health care establishment to be used as a base line for medical information of users of the various system embodiments described herein.

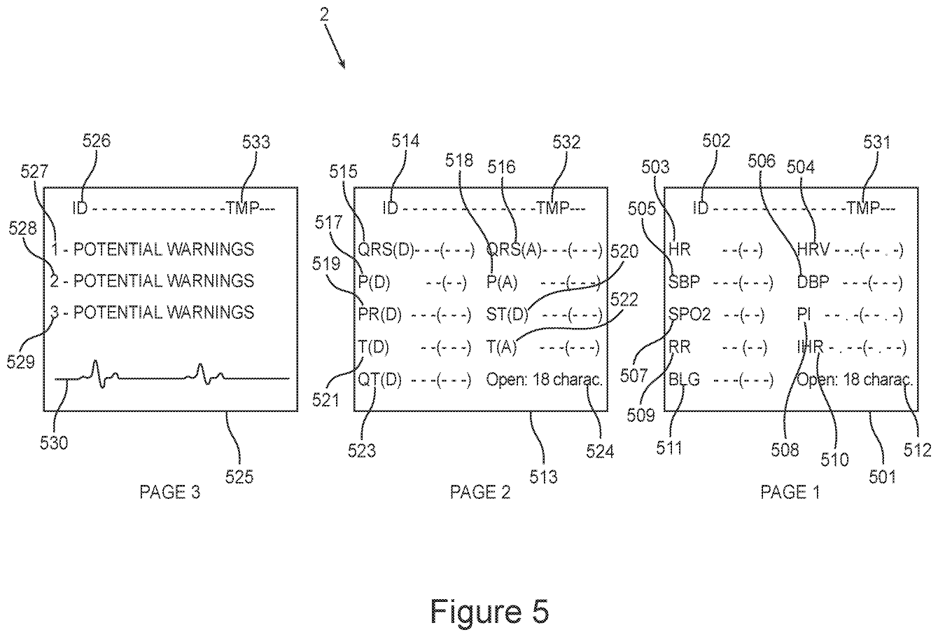

Processing unit 2 in at least certain embodiments may include a power supply for the recording unit's operation. Such power supply may also be used for charging and operation of sensor unit 1. Processing unit 2 is described in FIG. 5, 6 and as part of FIGS. 7 to 9.

FIG. 1 depicts a block diagram of an embodiment of a sensor unit 1 suitable for providing information from three types of sensors.

A first one of the sensors may be a Temperature sensor 101 possibly based on a high accuracy NTC type of sensor (0.1 Celsius degrees in between 30 to 42 degrees) or the like. Sensor 101 may be located within a heat conductive, corrosion proof metal capsule with thermal coupling paste.

A microcontroller (MCU) 105 may be provided within sensor unit 1 together with circuitry suitable for Analog to Digital conversion of data/information obtained by sensor within sensor unit 1 for use in the MCU. MCU 105 may be arranged for processing and applying calculations according to algorithm in order to provide numerical results of e.g. the measured temperature.

A PPG (Photoplethysmogram) sensor 102 may be based in one example on reflective type of PPG (in contrary to transmission type). PPG sensor 102 in one example may include two Green transmitting LED (λ peak 526 nm), one Red LED (A peak 660 nm), a Photodiode receiver for Red and Green lights, one IR (λ peak 950 nm) and IR dedicated Photodiode receiver. Sensor unit 102 may be divided into three compartments in order to prevent unwanted leakage of light.

In between PPG sensor 102 and the AFE within MCU 105, a protection circuit 107 may be provided in order to prevent damage to the MCU in case of a failure in the sensor unit (e.g. due to a shorted LED). The information from the sensor may be transferred to the AFE (Analog Front End) in the MCU 105.

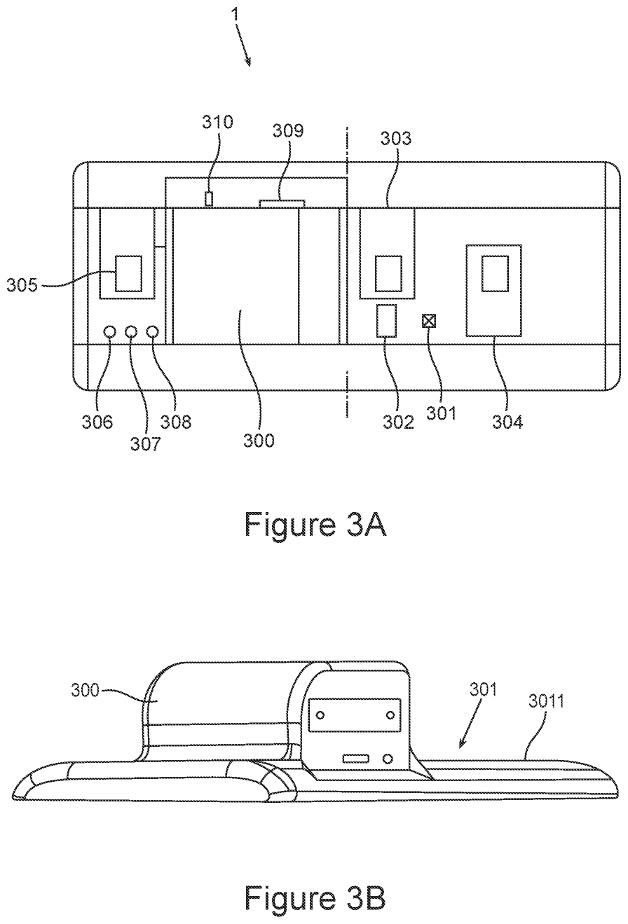

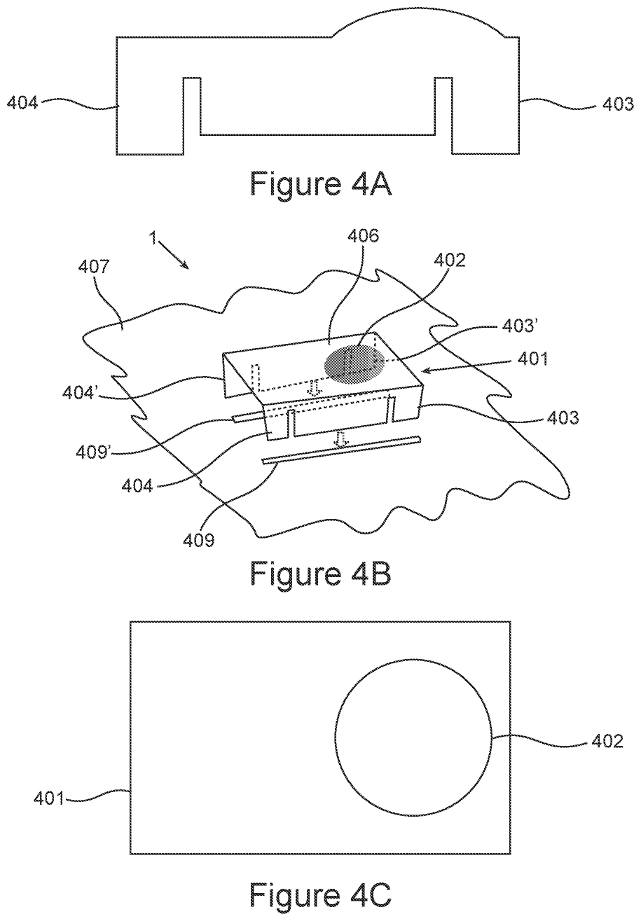

ECG (Electrocardiogram) sensing may be provided by three electrodes as detailed in FIG. 1 where numeral 104 indicates one electrode and numeral 103 indicates two electrodes (in FIG. 3 the single one electrode is indicated by numeral 305, while the two electrodes are indicated by numerals 303 and 304). Electrode 104 in FIG. 1 (or 305 in FIG. 3), may be arranged for being touched by a left hand finger during a test interval. An interior aspect of the right hand wrist of a user may be seated on a dedicated area of sensor unit 1 housing two electrodes (accordingly 103 in FIG. 1 indicating two electrodes or 303 and 304 in FIG. 3 indicating same two electrodes). Said dedicated area may be arranged for ensuring a fine contact which provides low noise, and detailed results to the AFE. Such dedicated area for the wrist region may be embodied as the dome part 402 seen in FIG. 4.

Two electrodes 103 may be located on the top right part of the sensor unit 1. One Electrode may be suited for the right hand which closes a measurement circuit of Electrocardiogram provide by the left hand finger. The second electrode may accordingly be arranged for contact with an interior section of the right hand wrist to serve as the reference electrode. An RLD (Right Leg Drive) circuit may be used in order to provide the suitable detailed and low noise information, which can be achieved from two electrodes ECG measurement system.

In between electrodes 103 and 104 and the AFE with MCU 105, a set of electronic dedicated components 107 may be arranged for the managing of the impedance matching and filtering in order to provide the best available noise free, good differential and impedance matched signals from the electrodes 103+104 to the AFE part of MCU 105.

AFE in MCU 105 may be arranged for receiving the information from both PPG 102 and ECG 103+104 sensors. The AFE device may be an analog front-end (AFE) for synchronized ECG and PPG signal acquisition. The PPG signal chain may support up the four switching LEDs and the two photoelectric receivers. The current from the PD may be converted into voltage by the trans-impedance amplifier (TIA) and digitized using an analog-to-digital converter (ADC).

The ECG signal chain may have an instrumentation amplifier (INA), with a programmable gain that interfaces to the same ADC. A right-leg drive (RLD) amplifier set can be used to bias the ECG input pins. AC and DC lead-off detect schemes can be supported. The ADC codes from the PPG and ECG phases can be stored in a 128-sample first in, first out (FIFO) block and read out is both in I 2C and SPI protocols.

The output of the AFE part in MCU 105, may be transferred to a processor (such as an Integrated 32 bit Arm Cortex-M3) and a transceiver (such as a 2.4-GHz RF transceiver)—in order to provide the management of graphical representations of a system of the present invention that may be provided as several plots (e.g. four plots).

Such plots may include the ECG, Red LED, Green LED and IR LED with the temperature measurement received from TMP sensor 101. The processor may send such plots to the main MCU calculating part and receive them back as a set of results.

The processor may then send the results due to a system protocol to processing unit 2, to any computer with a system software, local or connected by any art net and by the 2.4 GHz RF transceiver with on PCB Antenna, to any mobile device with downloaded subject system application. Low active RF and MCU current and low-power mode current consumption provide excellent battery lifetime.

In one example, an Arm Cortex-M7 device may serve as the main MCU, i.e. as the calculating part of a subject system embodiment. A set of several different algorithms (e.g. seven algorithms) may be loaded on such an Arm Cortex-M7 device. Each algorithm may be configured to use one or more of PPG LEDs plots, ECG electrodes plot or a combination of bot PPG LEDs and ECG results. Each such algorithm may be arranged to provide a single or group of results.

Such customized Algorithms may be arranged to provide several results, e.g. in one example provide eighteen medical/biological or more important and most useful parameters and written exemplary list of warnings in the case the ECG analysis finds results which require a warning as defined in the embodiment.

A list of parameters that such algorithms may be suited to calculate and show as digits may be described as exemplified below by the common short name of the parameter, description of parameter, the number of digits related to the parameter and the depict of the parameter digits, if required, in parenthesis:
HR—heart rate—2 digits.
HRV—Heart Rate Variation—4 digits: (2 digits, point and part of number digit)
SBP—Systolic Blood Pressure—3 digits
DBP—Diastolic Blood Pressure—3 digits
SPo2—Saturation Percentage of Oxygen level—3 digits
PI—Perfusion Index—4 digits: (2 digits, point and part of number digit)
RR—Respiration Rate—2 digits
IHR—Inhale Exhale Respiration ratio—4 digits (digit, point. 2 digits)
BGL—Blood Glucose Level—3 digits
QRSD—QRS duration (ECG)—3 digits (millisecond)
QRSA—QRS Amplitude-(ECG)—4 digits (mV, point, 2 parts of mV)
P(D)—P Wave duration-(ECG)—3 digits (millisecond)
P(A)—P Wave amplitude-(ECG)—4 digits (mV, point, 2 part of mV)
PR(D)—PR duration (ECG)—3 digits (millisecond)
ST(D)—ST Segment duration (ECG)—3 digits (millisecond)
T(D)—T Wave duration-(ECG)—3 digits (millisecond)
T(A)—T Wave amplitude-(ECG)—4 digits (mV, point, 2 parts of mV)
QT(D)—QT duration (ECG)—3 digits (millisecond)

The following exemplifies possible algorithms that may be implemented in various system embodiments in order to calculate parameters derived from the sensors and AFE, which may then be displayed in various type graphical representations of a system:
Algorithm A—MTP-HR, Calculates HR and HRV from both ECG and Green LED plots.
Algorithm B—MTP-BP, Calculates SBP and DBP from ECG and Green LED pulse transit time (PTT).
Algorithm C—MTP-SP, Calculates SPo2 and PI from Red and IR LEDs.
Algorithm D—MTP-RC, Calculates RR and IHR from ECG and all 3 LEDs plots.
Algorithm E—MTP-GL, Calculates BLG from ECG and all 3 LEDs plots.
Algorithm F—MTP-ECGP, Calculates all 9 ECG parameters from ECG plot.

One more algorithm, i.e. Algorithm G—MTP-ECGP, may be used to inspect Algorithm F results, Heart Rate, as well the level of ST line (Elevation/Depression compared to base line) and structure of P wave and similar wave structure which may not be defined in 9 measured parameters. The algorithm may pass over the list of the predefined warnings typical ECG characteristics and compare to the real time check. In the case where one or more predefined warnings characteristics match the test results, the written warning type (see list below) may be sent to the display of the system PAGE 3 as described in FIG. 5, as well to the other external connectivity as computers and mobile devices by the Bluetooth facility.
ST elevation
ST Depression
Supraventricular tachycardia
Ventricular Tachycardia
Bradycardia
Atrial Flutter
Atrial Fibrillation
Ventricular Fibrillation
Bundle branch block
AV block
Left bundle branch block
Right bundle branch block
Right ventricular hypertrophy
Left ventricular hypertrophy
Tachycardia
Hyperkalemia
Hypokalemia
Sick Sinus Syndrome

External DC power may be supplied to sensor unit 1 via the USB port 108 (see FIG. 1). The USB port may serve as just power supply connection in the case that sensor unit 1 is used as standalone device, sending information via internal Bluetooth facility. Embodiment of a complete system with processing unit 2 may require the data transmission from sensor unit 1 to processing unit 2. Such data transmission may be transferred via USB port to sensor unit 1 or any remote computer with the system display software. The external power supply may be arranged to charge the battery by the dedicated part of the internal power supply 109.

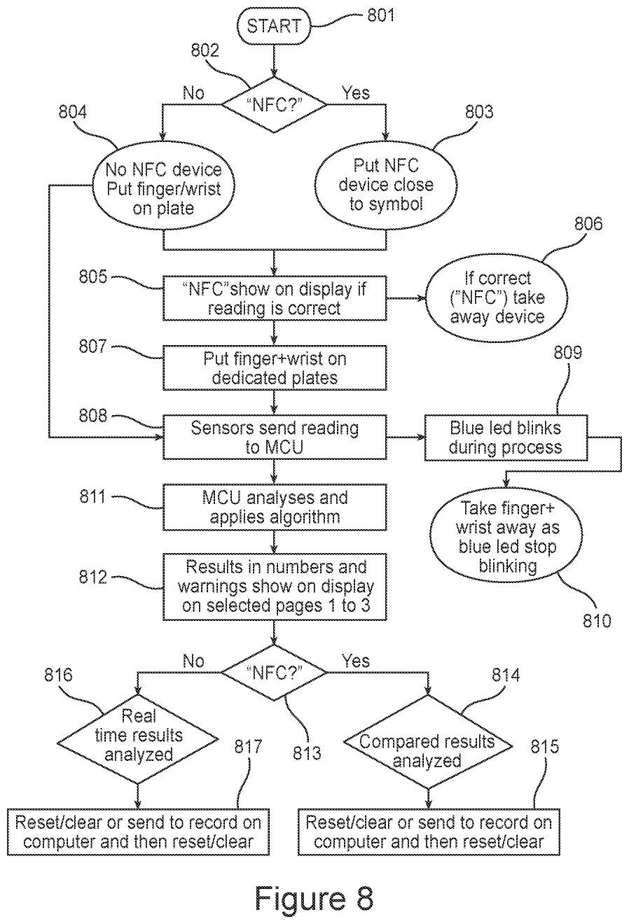

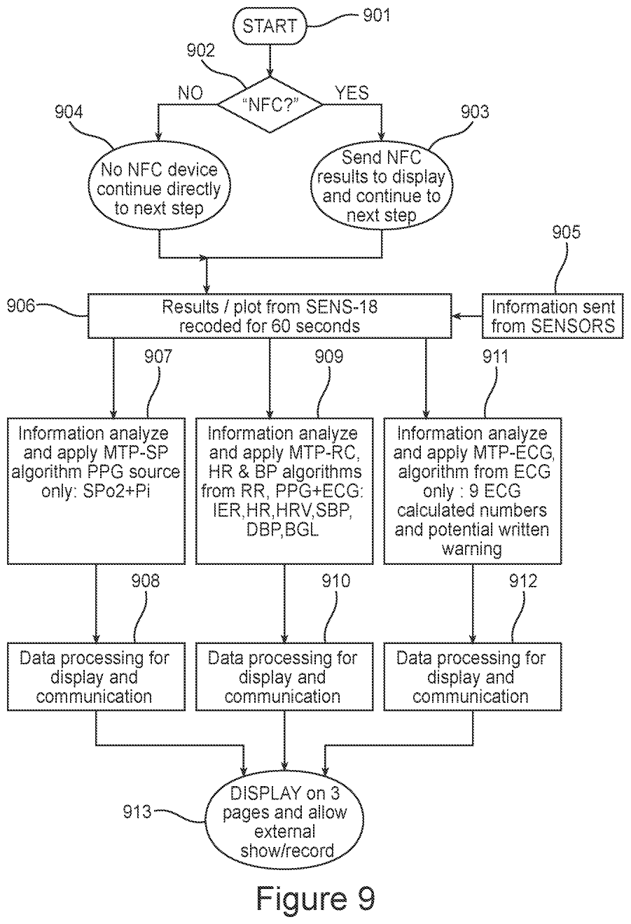

Embodiment of the ECG sensors in medical grade, require very low noise power source. In order to achieve the required power source and to ensure long time of self-operation, e.g. a 3.7V 600 mAH Lithium Ion standard Battery may be assembled in sensor unit 1. Several voltages may be demanded for the few different electronic devices included in sensor unit 1, therefore, a dedicated local power supply 109 assembled on the main PCB, supplying the different voltages as required by the several components sensor unit contains. FIG. 8 in components 808 to 811 and FIG. 9 in components 905 to 912 describe AFE/MCU part of sensor unit 1 as part of system flow chart.

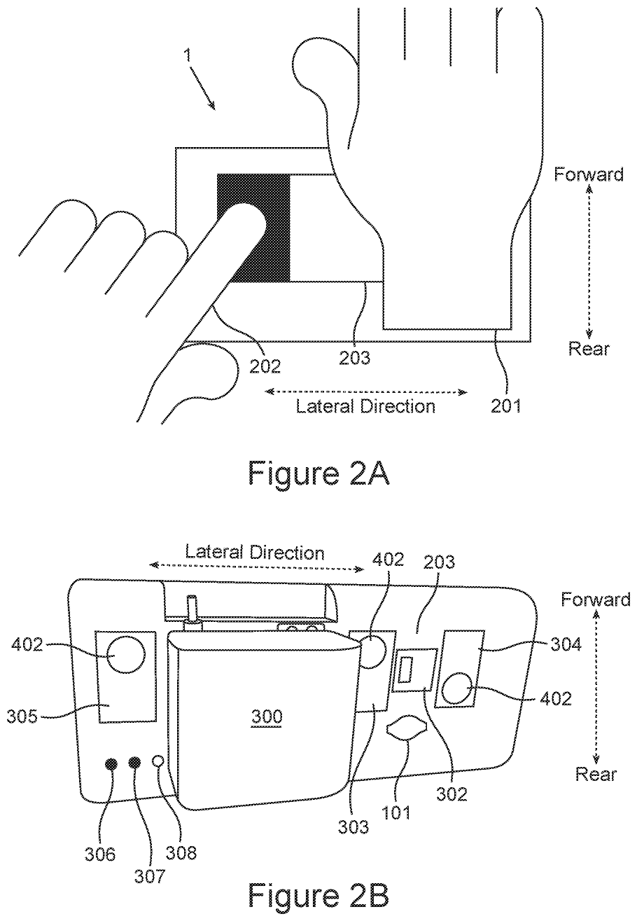

FIG. 2A illustrates, according to embodiments of the subject system, the way a user may place with his/her fingertips leading in a forward direction, an interior aspect of the right hand wrist 201 and left hand middle or index finger 202, on respective lateral left and right hand sides of a top part 203 of sensor unit 1. See opposing forward and rear directions and the generally orthogonal lateral directions indicated in the figure. Notably, certain sensors unit embodiments (not shown) may be arranged to locate the discussed electrodes/sensors at opposing lateral sides for accommodating an interior aspect of the left hand wrist and right hand middle or index finger in order to sense and derive the sets of parameters disclosed herein.

FIGS. 3A and 3B depict, respectively, exemplary over view and perspective views of sensor unit 1. An exemplary enclosure of sensor unit 1 may be made of Acrylonitrile Styrene Acrylester and Polycarbonate.

A tower like part 300 of the top cover, possibly 24 mm high in a non-binding example, may serve as a stopper for the inner side of Interior aspect of the right hand wrist. Tower 300 may be arranged to contain the LI battery incorporated in subject embodiment with a possible approaching door, optionally locked with 2 screws from back side of the enclosure.

A top part 301 of the temperature sensor 101 may be built to include a small dome (see example of dome 402 in FIG. 4) with its top apex reaching e.g. about 1.5 mm over an otherwise generally flat surface 3011 of top part 301. Said dome may be suited for ensuring a good thermal contact to the skin.

PPG sensor 302, possibly containing four light transmitting LED's at three wave lengths and two receiving Photodiodes, may be located flat or flush at the surface level.

Right hand ECG electrode 303, RLD reference electrode 304 and Left hand electrode main surfaces, may be located at surface level of top part 301. Dome 402, as described in FIG. 4, ensures suitable contact to the detected skin. The location of the two electrodes has been optimized by conducting a research including above sixty subjects in the ages from 5 years to 72 years old, different genders and skin colors. Left hand finger electrode 305 can be located at the left top part of sensor unit 1.

Green LED 306 may indicate a fully charged battery and may serve as an “ON” indicator LED in normal conditions.

Red LED 307 may indicate charging condition of the incorporated battery 110, managed by inner power supply 109.

Blue LED 308 may indicate the testing process. It may be arranged to blink during successful test procedure and light up constantly e.g. for more than about fifteen seconds after test complete, and then turn off until next test starts.

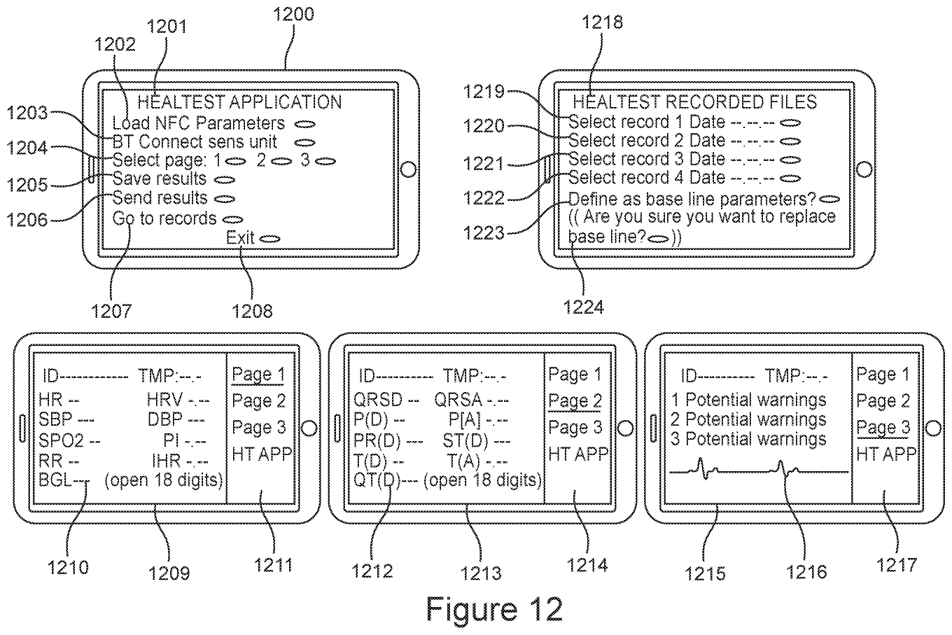

USB connector 309 may serve as the communication port towards processing unit 2 or any other computer device. USB connector 309 may also serve as a 5V 2 A DC power supply input in a standalone operation, normally by using Bluetooth communication to a mobile device or other Bluetooth equipped device with subject system software as depicts in FIG. 12.

Switch 310 may serve as sensor unit's power switch. Switch 310 may be connected in series with the incorporated battery 110.

FIGS. 4A to 4C depict a views of an embodiment of an electrode 401 developed for the subject system. Electrodes may be made of Stainless Steel alloy with special conduction processing. Three such electrodes can be installed on a top part 407 (see also top part 301/3011 marked in FIG. 3) of an enclosure of a sensor unit embodiment and serve as the ECG detecting electrodes in the subject embodiment. The electrode example 401 shown in FIG. 4 is one which comprises a dome, however electrodes, such as that suitable for functioning as electrode 305, may have a generally other structures, for example not necessarily comprising a dome.

The design of the electrode 401, which in this example optionally includes dome, may enable a high level of contact due to e.g. a 10 mm or the like round, built in dome part 402 which can be raised, e.g. by about 1.5 mm or the like, over the electrode's top panel surface level 406.

Due to signal to noise consideration, an optimal total surface area of electrode 401 may be desired. Achieving such optimal area size while keeping practical sizes on enclosure surface and proper distance in between Right hand and RLD electrodes, may dictate a suitable design. In an example, top part 406 size may be about 24 mm long and about 15 mm wide and an added area may be provided in this example by an about 90 degrees downwards folding that may be formed at opposing lateral sides of the Stainless Still part 401. The added folded lengths (see downward parts 403 and 404) demonstrate a possible manner in which such optimized total area of the electrode may be obtained.

Ensuring a robust and heavy duty assembly of sensor unit 1, two downwards parts 403 and 404 of the electrode sheet 401, and two parallel parts 403′, 404′ on the other side of the electrode 401 may be designed. Attaching electrode sheet 401 to sensor unit 1 (see FIG. 4B) may be accomplished by inserting the folded parts 403, 404 and their parallel parts 403′, 404′, into respective slits 409, 409′ formed through top part 407 of the enclosure of sensor unit 1 and then possibly folding up the parts 403, 404, 403′, 404′ towards the lower side of top part 407 of the sensor unit's enclosure, to thus ensure a strong positioning of electrode sheet 401 to the sensor unit in a manner enabling also simple replacement of the electrode sheet 401 if later required (e.g. due to maintenance or the like).

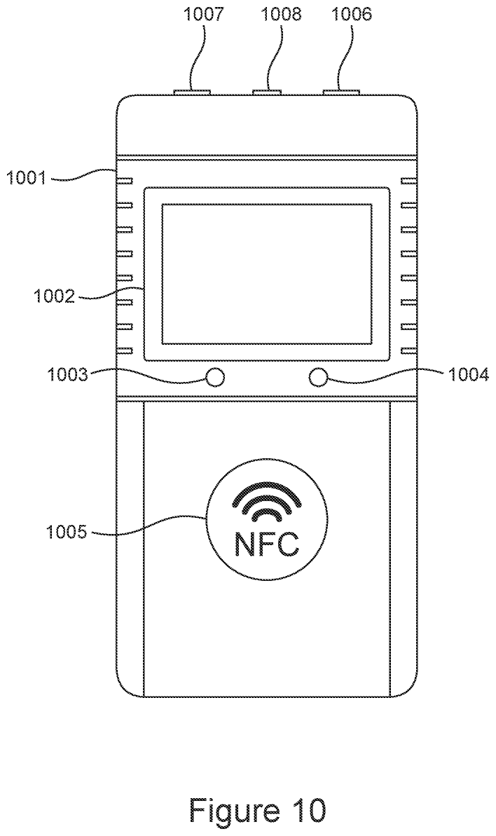

FIG. 5 depicts a possible graphical representation of a system here including three pages as sent from sensor unit 1 and displayed in processing unit 2, which may be an external computer with system software, a mobile device with system application (or the like). In large screen computer, all pages may be presented in one screen, while both in processing unit 2 and a mobile application, it may be required to display each page in a different screen. Using processing unit 2, selecting of a page may be executed by pressing a dedicated switch 1004 (see FIG. 10). Using computer or application, on the bottom of each page displayed, all pages names may be represented as a selector switch as known in art: [Page 1] [Page 2] [Page 3].

On top of each page 20, identity “ID” characters may be enabled as indicated in FIG. 5 by numerals 502, 514 and 526. ID characters may be free configurable fields for a user according to his/her local administration and needs. Examples may include national given ID number, internal health care body ID number (etc.). It may be recommended to use the three last characters for the blood type of the ID owner and to include recording or test date.

Sensor unit 1 provides patient temperature as measured by sensor 101 which may be presented on each top page row, after the ID information, as marked by numerals 531, 532 and 533. In the case where processing unit 2 may be used only for NFC information, the temperature may not be presented as it is only a real time parameter.

Each row may be presented in different color, enabling fast and convenient reading of results. Two parameters may be presented in a single row as see in this example. Each parameter, from left to right may be defined by the parameter short common name then real time results as derived from sensor unit 1 and last are the base line data in this example in parentheses as derived from the NFC device 603.

Page 1 that is marked by numeral 501 may be used for presenting nine parameters in five rows. In each row, the presented parameters may be related to each other. Parameters in the first row in this example represent Heart Rate related parameters HR 503 and HRV 504. Parameters in the second row represent in this example Blood Pressure related parameters SBP 505 and DPB 506. Parameters in the third row represent in this example Oxygen level in blood related parameters SPo2 507 and Pi 508. Parameters in the fourth row represent in this example Respiration related parameters RR 509 and IHR 510.

Parameters in the fifth row represent in this example Glucose (sugar) level in blood marked as BGL 511. This parameter may be defined by the users preferably in tests performed at constant time gap from meals. The right section of the fifth row may be as seen in this example an open field for NFC important recorded information regarding the specific patient. Such open field 512 may enable 18 characters. Possibly, the BMI of the patient may be recorded as part of the open field 512 or as part of ID (see numerals 502, 514 and 526).

Page 2 indicated by numeral 513 may present nine main parameters as a result of calculating the ECG plot, derived from sensor unit 1. Parameters show duration of a parameter, marked (D) and as well amplitude of part of the parameters, marked (A). First parameters row represents the QRS interval as QRS(D) 515 and QRS(A) 516. Second parameters row represents the P interval as P(D) 517 and P(A) 518. Third parameters row represents the PR interval as PR(D) 519 and ST interval as ST(D) 520. Forth parameters row represents the T interval as T(D) 521 and T(A) 522.

Fifth parameters row represents the QT interval as QT(D) 523. The right section of fifth row is an open field 524 for NFC important recorded information regarding the specific patient related to patient hearth condition or directly to ECG. The open field enables 18 characters.

Page 3 indicated by numeral 525 may represent potential warnings as a result of applying algorithm on both the nine numerical parametric results and as well directly on the ECG plot. Warnings can be taken from a predefined list of potential problems directly related to the ECG plot curve or from comparing parameters and plot to a least of symptoms. Warnings, if any, may be displayed in this example in the three open rows 527, 528 and 529. Each row may contains thirty characters. Below the three warning rows the real time ECG plot as received from sensor unit 1, may be displayed.

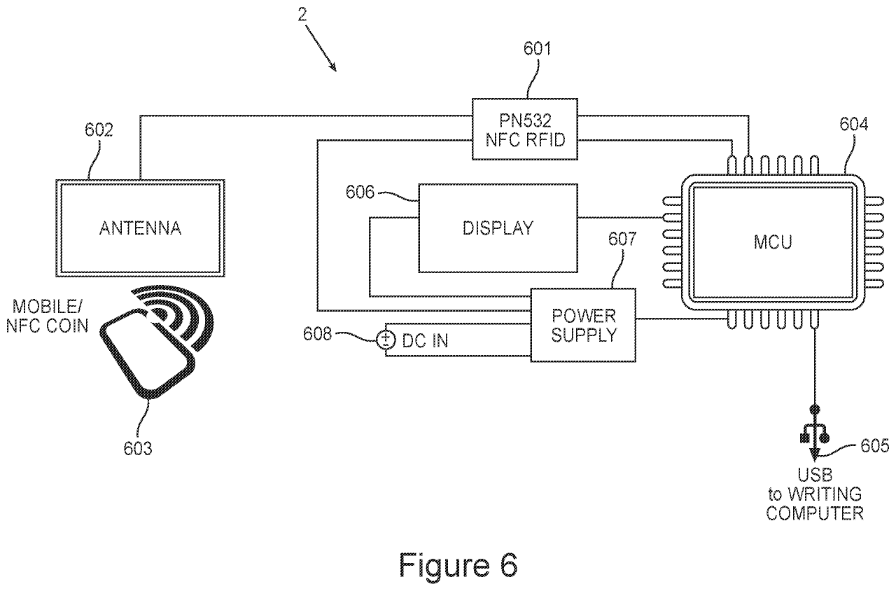

FIG. 6 depicts the use of processing unit 2 as a writer or reader only device of the selected eighteen parameters due to the subject system embodiment. A reader such as the 13.56 MHz, NXP NFC chip 601 can provide the NFC RFID management system. MIFARE Classic, Fully compliant with ISO/IEC 14443 Type-A protocol with 4 kB memory and 7 bytes in one example may be used in present embodiment of the subject system.

Embodiment of the system base line data recording can be based on Near Field Communication (NFC) which refers to short-range, high frequency wireless communication technology. RFID technology is a subset of NFC. NFC is an extension of the ISO 14443 proximity-card standard that combines the interface of a “smartcard” and a reader into a single device. NFC devices communicate via magnetic field induction, where two loop antennas are located within each other's near field, effectively forming an air-core transformer.

It operates within the globally available and unlicensed radio frequency Industrial, Scientific, and Medical (ISM) band of 13.56 MHz, with a bandwidth of almost 2 MHz, NFC technologies are deployed in wireless communication mobile stations (e.g., mobile phones, PDAs, laptops, and the like), primarily for public transportation ticketing and debit/credit payment transaction applications. Radio Frequency Identification (RFID) devices are well known in the art and widely deployed, e.g., as identification tags. In its simplest form, a passive RFID device includes an RF antenna and a simple electronic circuit.

The circuit ma be powered by a small electrical current induced in the antenna in the presence of an RF carrier wave transmitted by an RFID reader. The RFID device transmits data, such as its unique identifier, by backscatter modulating the RF carrier wave. The RFID reader detects the backscatter modulation, and recovers the identifier transmitted by the RFID device. Such RFID “tags” are widely used for inventory control and supply chain management, and are expected to replace optical codes such as “bar codes” on consumer products packaging, document courier envelopes, and the like. Active RFID devices include a power source, such as a battery, and often include more sophisticated circuits (e.g., processor and memory) and a full transceiver capable of two-way communication with an RFID reader.

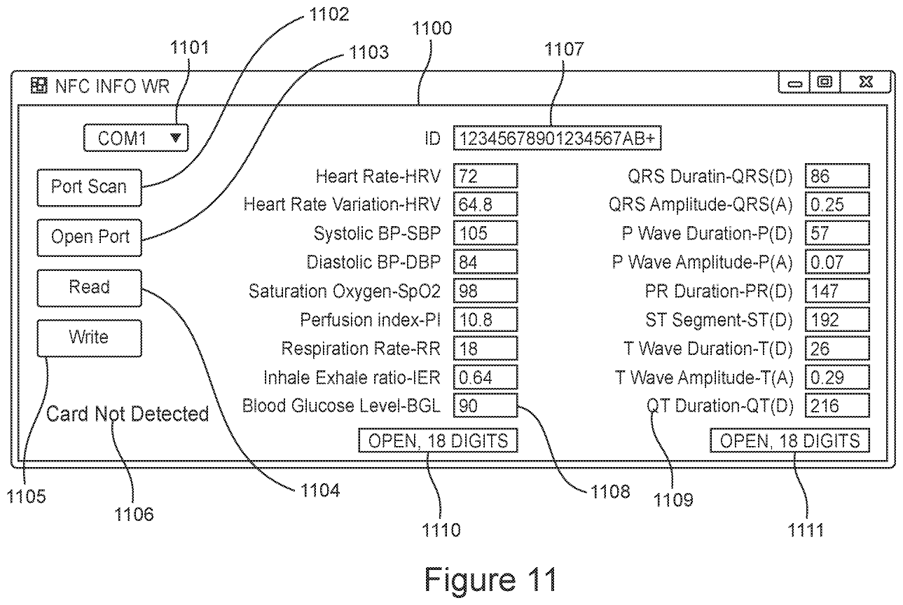

Recording of the ID and parameters data shall be done by a computer connected to processing unit 2 via the USB type of input/output connectivity 605. Data to be recorded on the selected NFC device may be provided by gold standard medical test equipment the user has the approach to. Data can be located as well from existence files or any other source known to user. User shall then manually insert the parameters numbers into the dedicated space as depict in FIG. 11.

As well, the Data can be automatically located from a sensor unit 1, connected via another USB port to the recorder computer. I certain cases, the ID details which may be inserted manually to the right space on screen may be excluded. Software, dedicated to subject system shall be downloaded to the recorder computer and manage the recording procedure as described in FIG. 11.

Mobile Phone with NFC facility or Coin types of NFC devices may be used as the recording media. The selected recording device 603 may be placed on the flat area of processing unit 2 possibly centered over the NFC mark 1005. The NFC mark 1005 cane be located over the center of the NFC RFID dedicated antenna 602, enabling the Wireless communication in between the recording device 603 and the NFC chip 601.

Antenna 602 may act as transmitter while recording on selected device 603 shall be executed, or, as a receiving antenna while reading data from when device 603 with data is faced or closely allocated over the mark 1005. MCU of Device B 604 serves as the manager device for all functions provided by processing unit 2. MCU shall receive the incoming data from the recorder computer, or, sensor unit 1 when a full system operation may be implemented. As well MCU 604 may send and receive the data from NFC chip 601 and send the configurator data to the display 606. Power Supply section 607 handles the incoming supply voltage from an external 12 VDC 2 A power source via a DC input connector 608. Power Supply section 607 shall stabilize incoming power and supply the dedicated power to MCU 604, NFC chip 601 and display 606.

FIG. 7 depicts the use of sensor unit 1 and processing unit 2 as a complete system, representing a real time test results compared to base line data which are both represented on display 706. The MCU complex 704 is represented as one block and includes the AFE and sensors calculating part 105, the Data connection via port 108 in sensor unit 1 to port 605 in processing unit 2 and then to the NFC chip 601+display 606 managing MCU 604.

Patient NFC device 703 represented over mark 1005 and antenna 702 may transmit the read Data the MCU part 704 via NFC chip 701 as detailed in FIG. 6. A patient may put his Interior aspect of the right hand wrist and left hand finger, as detailed in FIG. 2. Sensors complex 709, 710 and 711 may provide the tested data to MCU part 704. After 60 second period of data recording and analyzing, results will be represented aside base line as detailed in pages 1 501 and page 2 513 as detailed in FIG. 5. Page 3 525 shall represent the ECG plot relevant data while it is available, therefore, during the test period; it is recommended that Page 3 indicated by numeral 525 may be on display. Power supply 707 represents a general block, handling DC input 708 power source, dividing the dedicated power to all power consumption parts of the system and includes the battery implemented in sensor unit 1. Detailed flow chart of FIG. 8 and FIG. 9 depicts FIG. 6 on its right branches 803, 814, 815 and 903 respectively.

FIG. 8 depicts the flow chart of sensor unit land processing unit 2 used as a complete system, as depicts in FIG. 7 technical block diagram. As well, the left branch of FIG. 8 depicts the use of sensor unit 1 without NFC data provided as base line data.

START 801 of a new procedure may be enabled after a reset of previous check had been executed by pressing switch 1003. Mode of operation may be defined at next stage automatically 802.

If an NFC device 703 may be available, a patient or user may put or bring devices 803 and 703 substantially close to each other, possibly not more than 5 cm to marked symbol 1005. Device B may automatically react 805 due to presence of a NFC loaded device, if reading is correct.

Page 1 indicated by 501 may be presented automatically on display 706. I certain cases, in order to detect the full reading, it may be recommended to use switch 1004 and ensure page 2 (indicated by numeral 513) is correctly and fully loaded. NFC reading may stay on display due to internal memory of MCU 604.

When correct reading is ensured, NFC device may be released away from 1005 location 806. As reading is completed (normally during few seconds), patient may put his/her Interior aspect of the right hand wrist and left hand finger 807.

In the embodiment of subject system without the presence of a NFC device, Patient may put his Interior aspect of the right hand wrist and left hand finger 802. Processing unit 2 may automatically operate in the “no NFC base line data” mode.

As a result of both stage 804 and 807, 60 seconds test on sensor unit 1 may be executed, sensors shall send 808 their reading to the AFE/MCU 105. During the 60 seconds of successful test, as a result of MCU 105 command, the Blue high power LED 308 will blink 809, at end of the test period; the Blue LED will light constantly for more 15 seconds and then stop. During entire test period, the ECG lot may be directly transmitted and presented on Page.3 525.

Main calculating MCU 105/704 may starts to analyze and apply 811 in this example the seven installed Algorithms and the Temperature results reading from 101/709 after 30 seconds from start of a successful test. At the end procedure of calculating results 812, parameters in number and warnings in words, may be sent from sensor unit 1 to processing unit 2 and a command to stop blinking then constant light may be sent 809 to the Blue LED 308. As the Blue LED stops blinking, the patient may take away his/her hands from processing unit 2 and test procedure ends 810.

Due to selected mode, with or without NFC base line data 813, the parameters and warning may be presented. In a full system operation mode, both base line parameters with ID information, real time test results parameters, ECG final plot and potential warnings, shall be presented 814 on display 706 as depict in FIG. 5. Embodiment of the absence base line data mode 816 shall result in representing just the real time parameters, warnings and final ECG plot on display 706. In both modes, as evaluation of results finished, with or without recording of results in a remote device, 815 or 817, user shall press reset switch 1003 in order to clear internal memory of MCU 604 display 706.

FIG. 9 depicts a detailed flow chart of the analyzing and calculating part of the subject system. FIG. 8 depicts operational procedure, while FIG. 9 relates to the inner activity of the MCU.

START 901 of a new procedure shall be enabled after a reset of previous check had been executed by pressing switch 1003. Mode of operation shall be defined at next stage automatically 902.

NFC device 703 is available 903, recorded Data shall be sent to the display 706. Then, as patient start the test Sensors shall send the information 905 to the MCU via the AFE stage.

In the embodiment of subject system without the presence of a NFC device 904, as patient start the test Sensors shall send the information 905 to the MCU via the AFE stage.

Sensors results shall be recorded for 60 second 906. Exclude shall be the temperature r parameter which is simply measured after 30 seconds from test start, at one shot and will appear directly on display as 531, 532 and 533.

During about last 20 seconds of test and recording procedure 906 analyzing and calculating by applying the 7 dedicated will start. Part of algorithms will finish calculation during last 20 seconds of test procedure 906; in one example those may be algorithm C (AKA: MTP-SP) calculating SPo2 and Pi parameters from PPG 907 and algorithm B (AKA: MTP-BP) calculating SBP and DBP from both PPG and ECG sensors 909.

Remaining parameters and potential warnings, shall be calculated during 15 seconds after all 60 seconds of test completed; Based on both PPG and ECG data, algorithm D (AKA: MTP-RC) shall calculate RR and IER, algorithm A (AKA: MTP-HR) shall calculate HR and HRV and algorithm E(AKA: MTP-GL) shall calculate BGL 909. Based on ECG recorded data, algorithm F (AKA: MTP-ECGP) shall calculate the 9 selected ECG parameter, related to duration and amplitude of ECG segments Algorithm G (AKA: MTP-ECGW) shall analyze both parameters results and actual plot, comparing to a defined list of diseases symptoms, in order to reveal potential warnings 911.

As calculated data is ready, it shall be sent from the PPG only algorithm 908, from both PPG and ECG calculating algorithms 910 and from ECG only calculating algorithms 912.

Data presenting target shall be any of subject system 3 options;

Display 706 3 screens 501, 513 and 525 in Device B.
A mobile phone screens 1207, 1208 and 1209 with subject system application. Display shall be represented in the same format as in FIG. 5. Communication shall be executed by BT format using antenna 111. C) Any remote computer, loaded with subject system managing software. Display shall be represented in the same format as in FIG. 5. Communication shall be executed by USB connection 108.

FIG. 10 depicts an exemplary perspective view of the view over processing unit 2. Processing unit's 1001 exemplary enclosure can be made of Acrylonitrile Styrene Acrylester and Polycarbonate.

Processing unit 2 as detailed in FIG. 6 shall include a LCD TFT Display 1002. Display shall provide strong lighting and wide viewing angle, in order the user to see results from a distance of up to 1 m and from up to 30 degrees of axis.

Changing in between pages 1 to 3 shall be executed by pressing the momentary push button switch 1004.

Clear of current patient parameters and reset for the next patient use, executed by pressing the momentary push button switch 1003.

NFC device as Mobile Phone or Coin type, embodiment of writing into device, shall be used by placing the device on the plate, while device's antenna is over the area of the NFC symbol 1005. Embodiment of reading only of given NFC device shall be executed by placing device on the plate, or, just by bringing the device close to the NFC symbol 1005. A distance of up to 3 cm from symbol shall be enough to ensure proper reading of token data.

Connectivity to a writing computer loaded with subject system software, as described in present example shall be provided by a standard cable as known in common computers art. The connectivity socket on processing unit 2 marked 1006.

Connectivity to the external common market power supply shall be provided by a heavy duty DC socket 1008.

Connectivity to sensor unit 1, as described in present example, shall be provided by a standard 5 pins flexible and long lasting cable as known in common market. The connectivity socket on processing unit 2 marked 1007.

FIG. 11 depicts an exemplary perspective view of a computer screen 1100, ready to write data on a given NFC device which shall be laid on processing unit 1001, centered over the NFC symbol 1005.

Computer shall be connected to an operating processing unit and loaded with subject system software. As well, parameters can be typed manually.

Computer may have few communication ports. Several units of processing unit type, as well a direct connected sensor unit. Therefore, user shall select the right communication port 1101 due to a specific embodiment. As a relevant port had been selected and a relevant source had been found, color of “Port Scan” square will change, user then shall select “Port Scan” 1102.

As Port Scan 1102 had been selected, an indication of a proper format NFC device, or, an indication there is no proper format on port, shall be displayed 1106.

Shall the user decide to proceed with selected port due to the indication 1106 of a relevant NFC data; user may select “Open Port” 1103. Computer connected to sensor unit 1, shall not detect a card and software shall load directly the calculated parameters sensor unit 1 output provides in the dedicated screen squares. A NFC device, containing relevant formatted data, had been presented; ID, parameters and notifications shall be loaded into the dedicated squares.

A communication port had been opened successfully, user shall define required task; Reading 1104 the data from Device A, Device B or any other NFC source with subject system reading common format. Or, writing 1105 into NFC device laid on processing unit or any other NFC Reader/Writer device enabling subject system NFC common format.

Already loaded NFC device had been presented, patient ID shall automatically appear 1107. Initial writing information into connected device, requires the manual typing of patient ID 1107 and special notifications related to the specific patient, in the 18 open characters 1110 and 1111.

Set of Page 1 parameters and potential notifications, as described on FIG. 5, shall be automatically presented or manually typed on the left column of parameters 1108 and 1110. Set of page 2 parameters and potential notifications, as described on FIG. 5, shall be automatically presented or manually typed on the left column of parameters 1109 and 1111.

FIG. 12 depicts embodiment of subject system as an application used by a mobile phone 1200 or tablet device. System application shall support main common operating systems, Non-limiting examples of such operating systems are: Android, iOS, Chrome, Windows 10 Mobile, Blackberry 10 and Firefox OS. User shall down load system application (exemplary name “HEALTEST”). Selecting system's application on a mobile or tablet devices shall open managing main page 1201. The main page 1201 shall enable the user to select the other pages as display or for operation.

A given mobile device equipped with NFC section and loaded with subject system parameters, with or without ID and special owner notifications, user may load data from internal NFC facility, or from external presented NFC device, by selecting the relevant marked sign 1202. The information shall be represented automatically on Page 1 1209 and page 2 1213 in the dedicated location for base line parameters (in parentheses), as well the ID and patient special notifications if provided by the NFC device. Presentation of data matches the display as depicts in FIG. 5.

NFC data, internal or external, may not available, user shall see automatically base line parameters (possibly in parentheses), as well the ID and patient special notifications loaded from the defined base line files, pre-recorded on the mobile or tablet memory as per application. Selectin data group as a base line shall be done by selecting a specific recorded data in application recorded files page 1218, by selecting one of 4 recorded files 1219, 1220, 1221 or 1222. As one of the files had been selected and represented in the parentheses as temporary base line, the user shall be asked if presented record shall be defined as the base line 1223. If user decides he/she should use the presented record as a base line, in order to prevent occasional mistake, user may be asked to re confirm and make sure user desires changing present base line and replace it by presented record as a base line 1224. Selecting 1224, shall replace the base line. Decided user to select specific record as a new base line 1224, automatically selected record shall become record 1 1219 and other 3 records shall be arranged due to their recording dates as shown in 1220, 1221 and 1222.

User owns or has an approach to sensor unit of subject system, real time tested data can be loaded automatically by using a common art Bluetooth connectivity as provided by sensor unit. Loading data from sensor unit shall be executed by selecting sign 1203. Presentation of data matches the display as depicts in FIG. 5.

If a user were to desire to inspect data, such user may select pages 1, 2 or 3 1204. Each of the 3 data pages 1209, 1213 and 1215 divided into 2 sections;

Data section 1210, 1212 and 1216 as depicts in FIG. 5.
Page select section 1211, 1214 and 1217. Selecting enable inspect of pages 1 to 3 1209, 1213 and 1215, or, back to main page 1201.

Decides the user to save present test results data, user shall select 1205 and present data shall automatically become RECORD 4 1222, replacing present record marked as RECORD 4 1222 if there is already one.

Application enables user to send present displayed 3 pages 1209, 1213 and 1215 as files, to any other device with subject system application by selecting 1206. User may send the data for the evaluation of health expert as a medicine doctor or any other relevant establishment or person. Transmitting method of the current data shall be selected and executed by the specific options the user's device allows. Transmitting will be selected in the devices specific page which shall be opened due to selecting 1206. Data transmitting shall be made by any common available method as wired internet, Wi Fi internet, Wireless mobile communication or any other provided method.

Desires the user to inspect any of the 4 records in his device, or, select one of the records as the base line, user shall select 1207 on the main page 1201. Selecting one of 4 recorded files 1219, 1220, 1221 or 1222 the selected one shall be represented in the parentheses as temporary base line.

Desires the user to finish using subject system, user shall select EXIT 1208 and device's main page shall be displayed.

Attention is drawn to FIG. 2B illustrating an embodiment of a sensor unit 1 generally similar to that seen e.g. in FIG. 2A or 3A. In this view one can see the Right hand ECG electrode 303, RLD reference electrode 304, temperature sensor 101 and PPG sensor 302 located and exposed to a lateral right-hand side of a top surface of the sensor unit, while the Left hand finger electrode 305 is located and exposed to a lateral left-hand side of the top surface of the sensor unit.

Also seen are the possible Green, Red and Blue electrodes 306, 307, 308 that are located and exposed to the lateral left-hand side of the top surface of the sensor unit- and the tower like part 300 that serves as a stopper that divides between the first electrode at one of the lateral sides (here left lateral side) and the second and third electrodes and the PPG and temperature sensors at the other lateral side (here right lateral side) of the sensor unit.

In this example, the electrodes 303, 304 arranged to contact the interior aspect of the wrist are arranged to include each a dome 402 that projects above the outer top side of the housing of the sensor unit. A user may place his/her hands from above upon a top side of the sensor unit, with his/her fingertips leading in a forward direction-so that in this example the interior aspect of the right hand wrist and the left hand middle or index finger contact, respectively, the lateral left and right hand sides of the top side/part of the sensor unit. Optionally, such domes 402 as seen may be included in other electrodes/sensors, as here seen as being included also in electrode 305.

Experiments performed by the inventors have shown that at least in certain embodiments, optimal measurements at the interior aspect of the wrist may be obtained by positioning one of the domes 402, for example that of electrodes 303, in a more forward position relative to the other dome 402, for example that of electrodes 302, that is maintained at a more rear position. Such a more forward located dome may be arranged to contact the interior aspect of the wrist at a location that is more proximal to the radiocarpal joint of the wrist, while the other more rear dome may be displaced slightly rearward towards the user's forearm but still in a region of the interior aspect of the wrist.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

Further more, while the present application or technology has been illustrated and described in detail in the drawings and foregoing description, such illustration and description are to be considered illustrative or exemplary and non-restrictive; the technology is thus not limited to the disclosed embodiments. Variations to the disclosed embodiments can be understood and effected by those skilled in the art and practicing the claimed technology, from a study of the drawings, the technology, and the appended claims.

In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures can not be used to advantage.

The present technology is also understood to encompass the exact terms, features, numerical values or ranges etc., if in here such terms, features, numerical values or ranges etc. are referred to in connection with terms such as “about, ca., substantially, generally, at least” etc. In other words, “about 3” shall also comprise “3” or “substantially perpendicular” shall also comprise “perpendicular”. Any reference signs in the claims should not be considered as limiting the scope.

Although the present embodiments have been described to a certain degree of particularity, it should be understood that various alterations and modifications could be made without departing from the scope of the invention as hereinafter claimed.

1. A system for managing and testing medical data pertaining to a plurality users, the system comprising:
a sensor unit having a lateral longitudinal extension and comprising a housing and three electrodes, a Photoplethysmogram (PPG) sensor and a temperature sensor all housed within the housing and being exposed to an exterior upper side of the housing for obtaining sensed data representative of a medical condition each time from a given single user,
the sensor unit being of a tabletop type and the sensed data is obtained by:
a first one of the electrodes located at a first lateral side of the sensor unit being in contact with a finger of a first one of the hands of the given user,
a second one of the electrodes located at a second lateral side of the sensor unit being in contact with an interior aspect of the wrist of a second one of the hands of the given user,
a third one of the electrodes located also at the second lateral side of the sensor unit being an RLD reference electrode and being in contact also with the interior aspect of the wrist of the second one of the hands of the given user, and
the PPG and temperature sensors located also at the second lateral side of the sensor unit being also in contact with the interior aspect of the wrist of the second one of the hands of the given user, wherein
the sensor unit comprising a possible tower like part serving as a stopper that divides between the first electrode at the first lateral side and the second and third electrodes and the PPG and temperature sensors at the other second lateral side,
the system further comprising a processing unit for comparing the at least one measured parameter to base line data relating to the same given user, wherein
base line data is recorded on a peripheral device, such as a token device, a mobile phone or any other device including compatible NFC writing/reading facility, and wherein
the processing unit is adapted to gain access to the recorded base line data on the peripheral device for the comparison of the at least one measured parameter to base line data relating to the same given user.

2. The system of claim 1, wherein the given user being arranged to place his/her hands from above upon the sensor unit with his/her fingertips leading in a forward direction that is generally orthogonal to the lateral direction, while at least the second and third electrodes arranged to contact the interior aspect of the wrist of the given user each comprise a bulging member, for example a dome, that projects above the housing for contacting the interior aspect of the wrist.

3. The system of claim 2, wherein one of the bulging members is located at a more forward position relative to the other bulging member so that the bulging members contact the interior aspect of the wrist of the given user at respective forward and rear regions.

4. The system of claim 1, wherein the base line data comprises at least one previous measurement of the at least one parameter from the same given user.

5. The system of claim 4, wherein the base line data comprises identification (ID) information of the given user.

6. The system of claim 5, wherein the identification (ID) information comprises special personal notifications relating to the given user, e.g. known medical facts of the given user.

7. The system of claim 1, wherein the sensed data comprises sensing a middle or index finger of the left hand of the given user.

8. The system of claim 7, wherein the sensing of the middle or index finger of the given user is used for closing a measurement circuit, such as an ECG measurement circuit.

9. The system of claim 1, wherein gaining access comprises placing the peripheral device adjacent the system.

10. The system of claim 1, wherein gaining access is via any one of: wireless communication between the peripheral device and the system, at least one cable connecting the peripheral device and the system.

11. The system of claim 1, wherein the peripheral device belongs to the given user.

12. The system of claim 1, wherein the sensor unit and the processing unit are located at different locations for permitting sensing of medical condition of user(s) at one location to be reviewed/analyzed via the processing unit at another location.

13. The system of claim 1 and comprising a display for presenting the real time sensed data of the given user and/or the base line data relating to the same sensed data of the given user.

14. The system of claim 13, wherein the system being arranged for presenting on the display also warnings analyzed by comparison between sensed data and between base line data and/or real time sensed ECG data and/or sensed PPG data.

15. The system of claim 12, wherein presentation on the display is divided into several sections/pages, and wherein possibly in two sections/pages presentation is of real time sensed data in comparison to recorded base line parameters and in a third possible section/page presentation is of real time ECG plot and potential warnings derived and analyzed based on data provided by the ECG plot and parameters.

16. The system of claim 1, wherein sensed data is arranged to provide any one of temperature of a given user, ECG plot of the given user.

17. The system of claim 13, wherein the display is of a mobile device, e.g. a mobile cellular device.

18. The system of claim 1, wherein the sensor unit's housing being formed from a first material and each electrode comprises a second material that is different than the first material, and wherein the electrodes are attached to the housing.

19. The system of claim 18, wherein the second material is Stainless Steel alloy, possibly a coated Stainless Steel material.

20. The system of claim 18, wherein attachment of electrodes to the housing of the sensor unit is by forming openings in the housing, e.g. in form of one or more slits, and inserting at least a portion of each electrode into at least one of the openings in order to accomplish the attachment.

21. The system of claim 20, wherein at least some electrodes comprise each at least one folded section and attachment of such electrodes to the housing comprises inserting a folded section into an opening.

22. The system of claim 1 and being arranged to calculate and display physiologic parameters from sensed data obtained by the plurality of sensors, wherein the calculated and displayed physiologic parameters comprise any one of: HR (heart rate); HRV (Heart Rate Variation); SBP (Systolic Blood Pressure); DBP (Diastolic Blood Pressure); SPo2 (Saturation Percentage of Oxygen level); PI (Perfusion Index); RR (Respiration Rate); IHR (Inhale Exhale Respiration ratio); BGL (Blood Glucose Level); QRSD (QRS duration (ECG)); QRSA (QRS Amplitude-(ECG)); P(D) (P Wave duration (ECG)); P(A) (P Wave amplitude-(ECG)); PR(D) (PR duration (ECG)); ST(D) (ST Segment duration (ECG)); T(D) (T Wave duration-(ECG)); T(A) (T Wave amplitude-(ECG)); QT(D) (QT duration (ECG)).

23. The system of claim 1 and being arranged to calculate and display warnings derived from sensed data obtained by the plurality of sensors, wherein the calculated and displayed warnings comprise any one of warning-criteria: ST elevation; ST Depression; Supraventricular tachycardia; Ventricular Tachycardia; Bradycardia; Atrial Flutter; Atrial Fibrillation; Ventricular Fibrillation; Bundle branch block; AV block; Left bundle branch block; Right bundle branch block; Right ventricular hypertrophy; Left ventricular hypertrophy; Tachycardia; Hyperkalemia; Hypokalemia; Sick Sinus Syndrome, and wherein the warnings possibly comprise indications on deviations from medical standards regarding any of the warning-criteria.

24. A method for managing medical data pertaining to a plurality users, and comprising:
providing a tabletop type sensor unit having a lateral longitudinal extension and comprising a housing and three electrodes, a Photoplethysmogram (PPG) sensor and a temperature sensor all housed within the housing and being exposed to an exterior of the housing,
providing a processing unit,
obtaining by the sensor unit each time sensed data from a given single user by:
a first one of the electrodes located at a first lateral side of the sensor unit being in contact with a finger of a first one of the hands of the given user,
a second one of the electrodes located at a second lateral side of the sensor unit being in contact with an interior aspect of the wrist of a second one of the hands of the given user,
a third one of the electrodes located also at the second lateral side of the sensor unit being an RLD reference electrode and being in contact also with the interior aspect of the wrist of the second one of the hands of the given user, and
the PPG and temperature sensors located also at the second lateral side of the sensor unit being also in contact with the interior aspect of the wrist of the second one of the hands of the given user;
and
comparing at the processing unit between the sensed data of the given user and base line data relating to the same given user, wherein
the base line data comprises at least one previous measurement of similar sensed data of the same given user and identification (ID) information of the given user, and wherein
the comparing at the processing unit is performed only after obtaining sensed data by the sensor unit from a plurality of users.

25. The method of claim 24, wherein the sensor unit comprising a tower like part serving as a stopper that divides between the first electrode at the first lateral side and the second and third electrodes and the PPG and temperature sensors at the other second lateral side.

26. The method of claim 24, wherein the given user being arranged to place his/her hands from above upon the sensor unit with his/her fingertips leading in a forward direction that is generally orthogonal to the lateral direction, while at least the second and third electrodes arranged to contact the interior aspect of the wrist of the given user each comprise a bulging member, for example a dome, that projects above the housing for contacting the interior aspect of the wrist, wherein one of the bulging members is located at a more forward position relative to the other bulging member so that the bulging members contact the interior aspect of the wrist of the given user at respective forward and rear regions.

27. The method of claim 24, wherein the identification (ID) information comprises special personal notifications relating to the given user, e.g. known medical facts of the given user.

28. The method of claim 24, wherein sensed data comprises sensing a middle or index finger of the left hand of the given user.

29. The method of claim 24, wherein the PPG sensor comprises at least one of: two Green transmitting LEDs, one Red LED, a Photodiode receiver for Red and Green lights, at least one IR Photodiode receiver.

30. The method of claim 24 and being arranged to calculate and display physiologic parameters from sensed data obtained by the plurality of sensors, wherein the calculated and displayed physiologic parameters comprise any one of: HR (heart rate); HRV (Heart Rate Variation); SBP (Systolic Blood Pressure); DBP (Diastolic Blood Pressure); SPo2 (Saturation Percentage of Oxygen level); PI (Perfusion Index); RR (Respiration Rate); IHR (Inhale Exhale Respiration ratio); BGL (Blood Glucose Level); QRSD (QRS duration (ECG)); QRSA (QRS Amplitude-(ECG)); P(D) (P Wave duration (ECG)); P(A) (P Wave amplitude-(ECG)); PR(D) (PR duration (ECG)); ST(D) (ST Segment duration (ECG)); T(D) (T Wave duration-(ECG)); T(A) (T Wave amplitude-(ECG)); QT(D) (QT duration (ECG)).

31. The method of claim 24 and being arranged to calculate and display warnings derived from sensed data obtained by the plurality of sensors, wherein the calculated and displayed warnings comprise any one of warning-criteria: ST elevation; ST Depression; Supraventricular tachycardia; Ventricular Tachycardia; Bradycardia; Atrial Flutter; Atrial Fibrillation; Ventricular Fibrillation; Bundle branch block; AV block; Left bundle branch block; Right bundle branch block; Right ventricular hypertrophy; Left ventricular hypertrophy; Tachycardia; Hyperkalemia; Hypokalemia; Sick Sinus Syndrome, and wherein the warnings possibly comprise indications on deviations from medical standards regarding any of the warning-criteria.

32. The method of claim 24, wherein the managing of the medical data in on a mobile device.

33. The method of claim 24, wherein the processing unit is comprised in a mobile device and the managing of the medical data comprises comparing between sensed data and base line data on a downloadable software application on the mobile device.

34. The method of claim 24, wherein the displaying of physiologic parameters and/or warnings is on a downloadable software application running on a mobile device.
